(19) United States
(12) Patent Application Publication
(10) Pub. No.: US 2003/0176883 A1
(43) Pub. Date: Sep. 18, 2003

(54) Tissue manipulation apparatus and method of use

(76) Inventors: Jude S. Sauer, Pittsford, NY (US); Mark A. Bovard, Pittsford, NY (US)

Correspondence Address: Kenneth J. LuKacher, South Winton Court, 3136 Winton Road South, Suite 204, Rochester, NY, 14623, US

(21) Appl. No.: 10/095,842

(22) Filed: Mar. 12, 2002

(51) Int. Cl. A61M 29/00

(52) U.S. Cl. 606/198

(57) ABSTRACT

A tissue manipulation apparatus and method of use is provided to enable remote holding and multidirectional movement of tissue, such as the prostate gland to aid in its surgical removal during prostatectomy. The apparatus has a housing and a shaft extending from the housing which curves to a distal end. The shaft is insertable through a natural or surgical opening or channel in the body of a patient, such as the urethra of a male patient to access the patient's prostate gland. Tissue engaging members in the distal end are actuated by a longitudinal drive mechanism through the shaft from their retracted position at the distal end to extend into the tissue. The distal end is rotatable by a rotational drive mechanism extending through the shaft to the distal end. The longitudinal and rotational drive mechanisms are independently operated at the housing. The tissue once held by the tissue engaging members may be manipulated in its position under control of the operator of the apparatus, such as, raised or lowered by adjusting the tilt angle of the shaft, pulled or pushed by changing the extent of the shaft passing through the opening or channel to access the tissue, and bi-directional rotated via the rotational drive mechanism. In a prostatectomy, for example, the distal end is located in the prostate through the urethra and the tissue engaging members are extended. The operator can position the prostate to expose and place tissue surrounding the prostate under tension during dissection, while minimizing damage to surrounding tissue structures. The apparatus may further provide internal channels to facilitate the insertion or removal of liquids or gases from the site of tissue engagement. After dissection is complete, the tissue engaging members are retracted in the distal end to their retracted position via the longitudinal drive mechanism, and then the shaft and distal end is removed from the patient.

### FIELD OF THE INVENTION

The present invention relates to an apparatus and method for manipulation of the prostate, and relates particularly to an apparatus and method for manipulating the prostate to control the position of the prostate during prostatectomy. The invention is useful for manipulating a tissue structure accessible through a natural or surgical opening or channel in the body of a patient, and is especially useful to provide a surgical instrument which can be inserted through the urethra of a male patient to engage the prostate and enable a surgeon to move and position of the prostate, and thereby facilitate the dissection of the prostate during prostatectomy.

BACKGROUND OF THE INVENTION

Prostatectomy is a medical procedure to surgically remove the prostate gland of a male patient. The procedure is often performed due to disease of the prostate, such as cancer. The procedure may be performed by open surgery, perineally, or laproscopically by the use of endoscopic instruments through small incisions in the patient. In brief, the prostate is located along urethra leading to the bladder, and removal of the prostate is performed by exposing the prostate, dissecting the tissue surrounding the prostate, removing the prostate, and then suturing the urethra to the bladder. One problem often encountered during prostatectomy is that the prostate is difficult to position and maneuver by the surgeon to exposed the tissue and place the tissue under tension during dissection to extract the gland. This is especially a problem with laproscopic prostatectomy. Another problem is that the neurovascular bundles adjacent the prostate can be damaged during the prostatectomy negatively effecting normal penile functionality. Precise dissection is important to minimize damage to surrounding tissue and especially the neurovascular bundles. Therefore it would be desirable to provide an instrument which can be inserted through the urethra to engage the prostate and then enable the prostate's position to be manipulated during prostatectomy, thereby facilitate viewability and tensioning of tissue during prostate dissection.

Although transurethral medical devices have been developed, they are generally for use in urological procedures, such as catheter placement, and not for manipulation of the prostate.

SUMMARY OF THE INVENTION

Accordingly, it is an object of the present invention to provide a tissue manipulation apparatus capable of being inserted through an opening or channel in the body of a patient, and especially through the urethra of a male patient to engage and hold the prostate of the patient so as to control the position of the prostate during its dissection from the patient.

It is another object of the present invention to provide a tissue manipulation apparatus for engaging the tissue and enabling multiple degrees of freedom of movement of the tissue to the extent provided by other connecting tissue.

It is a further object of the present invention to provide a tissue manipulation apparatus having a shaft which is shaped to enable its insertion through a natural or surgical opening or channel to the tissue to be manipulated, such as being curved to enable insertion through the urethra of a male patient.

It is still another object of the present invention to provide a method for using the apparatus for positioning the prostate during prostatectomy to expose and place tissue surrounding the prostate under tension during dissection, while minimizing damage to surrounding tissue structures.

Briefly described, the present invention embodies an apparatus having a housing with a handle, a shaft which extends from the housing to a distal end and is curved near the distal end. The shaft is insertable through the urethra of a male patient's penis to locate the distal end in the patient's prostate gland, whereby the curvature of the shaft near the distal end enables insertion through the curved bony structure of the pelvis. Tissue engaging members in the distal end are actuated by a longitudinal drive mechanism through the shaft from a retracted position to extend into the prostate. The distal end is rotatable by a rotational drive mechanism extending through the shaft to the distal end. The longitudinal and rotational drive mechanisms can be independently operated at the housing. The prostrate once held by the tissue engaging members is positionable by movement (e.g., tilt, push or pull) of the housing coupled to said distal end by said shaft, or by rotation of the distal end by the rotational drive mechanism.

The invention further embodies a method for use of the apparatus to aid in the dissection of the prostate from a patient by the steps of: inserting the shaft and distal end through the urethra of a patient to locate the distal end in the prostate, extending the tissue engaging members from the distal end to hold the prostate onto the distal end, and positioning the prostate to expose and tension tissue to aid in the dissection of tissue enabling removal of the prostate from the patient, while minimizing damage to surrounding tissue structures, such as neurovascular bundles and the neck of the bladder of the patient. The positioning of the prostate is provided under control of the operator, e.g., surgeon, of the apparatus, such as, raised or lowered by adjusting the tilt angle of the shaft, pulled or pushed by changing the extent of the shaft passing through the urethra, or bi-directionally rotated. The extent of movement of the prostate being limited by other tissue connecting to the prostate. After prostatectomy is complete, the tissue engaging members are retracted into the distal end, the prostate is removed from the apparatus, and then the shaft and distal end are removed from the urethra of the patient.

The apparatus may optionally include internal channels along the shaft and distal end accessible from a port on the shaft to allow insertion or removal of liquids or gasses from the area or site where the distal end is located.

Although the tissue manipulated is described as the prostate and the apparatus for use in a prostatectomy, it may represent any tissue structure which is accessible through a natural or surgical opening or channel of a patient in other surgical procedures, where the shaft is shaped in accordance with the particular path through that opening or channel.

### BRIEF DESCRIPTION OF THE DRAWINGS

The foregoing objects, features and advantages of the invention will become more apparent from a reading of the following description in connection with the accompanying drawings, in which:

FIGS. 1A and 1B are perspective views of the apparatus in accordance with the present invention showing the tissue engaging members at the distal end of the apparatus retracted in FIG. 1A and extended in FIG. 1B;

FIG. 2 is an exploded perspective view of the apparatus of FIGS. 1A and 1B;

FIG. 2A is an exploded view showing the assembly of the tissue engaging members at the distal end of the apparatus of FIGS. 1A and 1B;

FIG. 2B is a cross-sectional view of the distal end of the apparatus along lines 2B-2B of FIG. 1A;

FIG. 2C is a cross-sectional view of the apparatus along lines 2C-2C of FIG. 1B;

FIG. 3 is a cross-sectional view along the length of the apparatus of FIG. 1A;

FIG. 3A is an expanded view of FIG. 3 broken away to show in more detail part of the longitudinal and rotational drive mechanisms of the apparatus;

FIG. 3B is an expanded view of FIG. 3 broken away to show in more detail one of the coupler member in the shaft of the apparatus;

FIG. 3C is a cross-sectional view along the lines of 3C-3C of FIG. 3;

FIG. 3D is a cross-sectional view along the lines of 3D-3D of FIG. 3;

FIG. 3E is a perspective view showing the assembly of the longitudinal drive mechanism of the apparatus of FIGS. 1A and 1B with the housing removed;

FIG. 3F is a perspective view showing the assembly of the rotational drive mechanism to the distal end of the apparatus of FIGS. 1A and 1B with the housing removed;

FIG. 3G is an exploded perspective view showing another embodiment for translation of rotation through the curved section of the shaft of the apparatus of FIGS. 1A and 1B;

FIG. 3H is a perspective view showing the coupling of embodiment of FIG. 3G to a rotational coupler near the distal end of the apparatus of FIGS. 1A and 1B;

FIG. 3I is a cross-sectional view of the curved section of the shaft of FIG. 3 in accordance with the embodiment of FIGS. 3G and 3H for rotation;

FIG. 3J is a cross-sectional view along the lines of 3J -3J of FIG. 3I;

FIG. 3K is a cross-sectional view along the lines of 3K-3K of FIG. 3I;

FIG. 3L is a cross-sectional view along the lines of 3L-3L of FIG. 3I;

FIG. 4 is a perspective view showing the shaft and distal end assembled with the longitudinal and rotational drive mechanisms of the apparatus of FIGS. 1A and 1B;

FIGS. 4A and 4B illustrate the operation of the longitudinal drive mechanism to actuate the tissue engaging members with the housing and shaft removed and the tissue engaging members retracted and extended, respectively;

FIGS. 4C and 4D illustrate an example of the operation of the rotational drive mechanism with the housing and shaft removed and the tissue engaging members extended and then rotated 90 degrees;

FIGS. 5A, 5B and 5C show a schematic of the male surgical anatomy in an example of the use of the apparatus of the present invention during a prostatectomy procedure;

FIG. 6 is a perspective view showing an embodiment of the present invention to provide a channel along the shaft to the distal end to allow insertion or removal of gases or liquids through a port to the shaft;

FIG. 6A is a cross-sectional view along lines 6A-6A of FIG. 6; and

FIG. 7 is a cross-sectional view similar to FIG. 3J for the embodiment of FIG. 6.

### DETAILED DESCRIPTION OF THE INVENTION

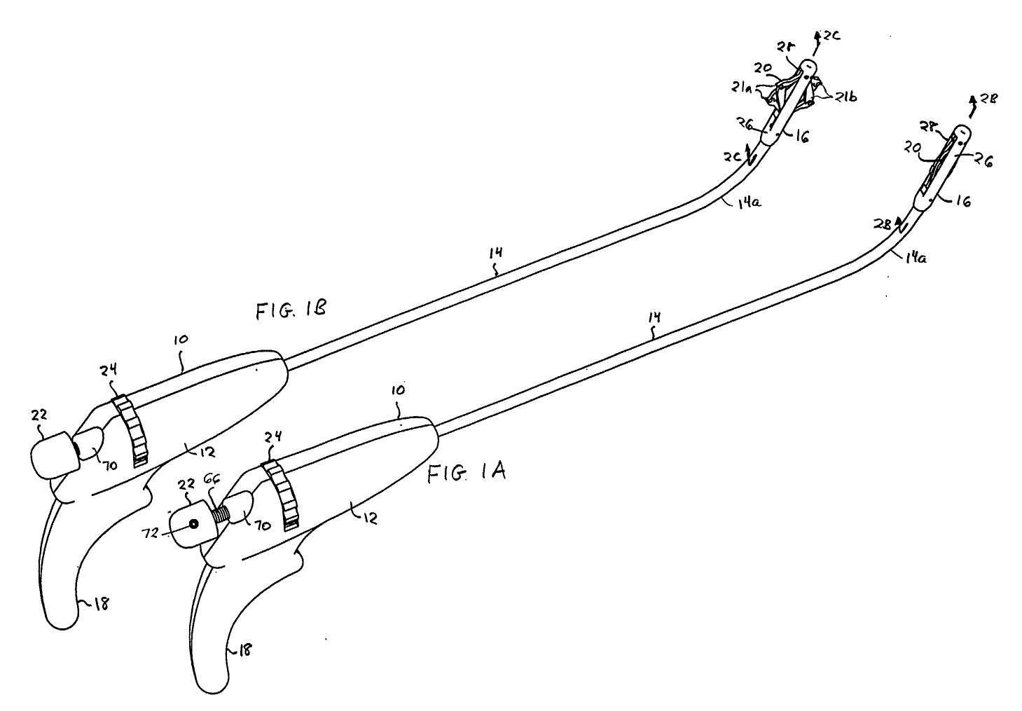

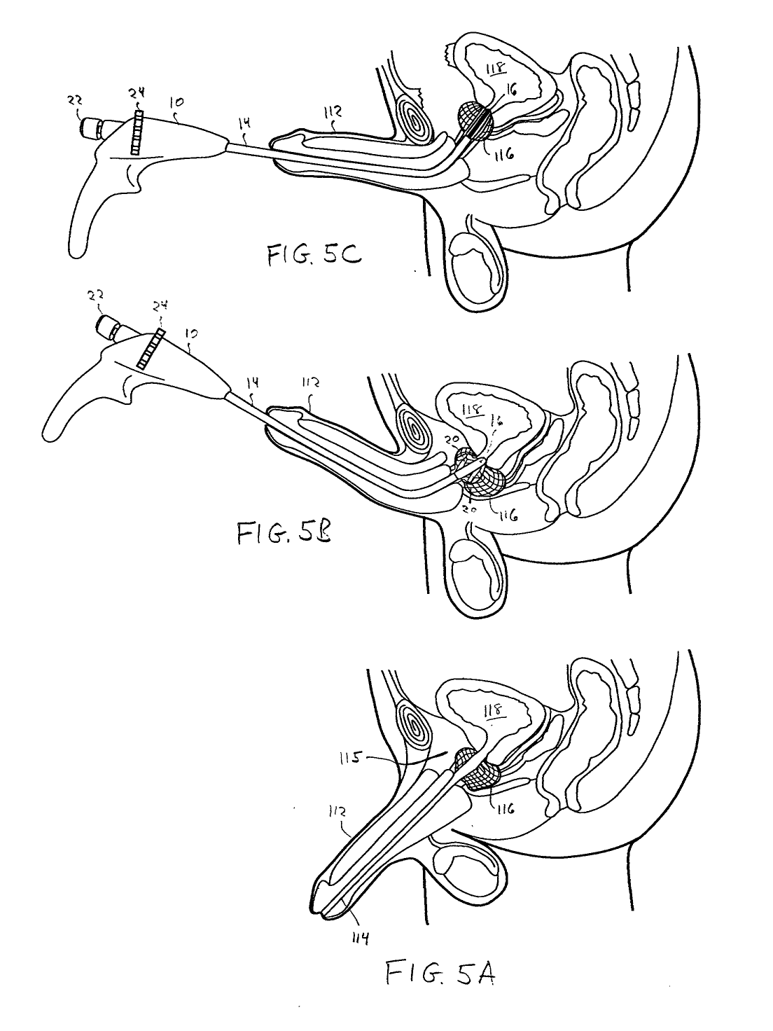

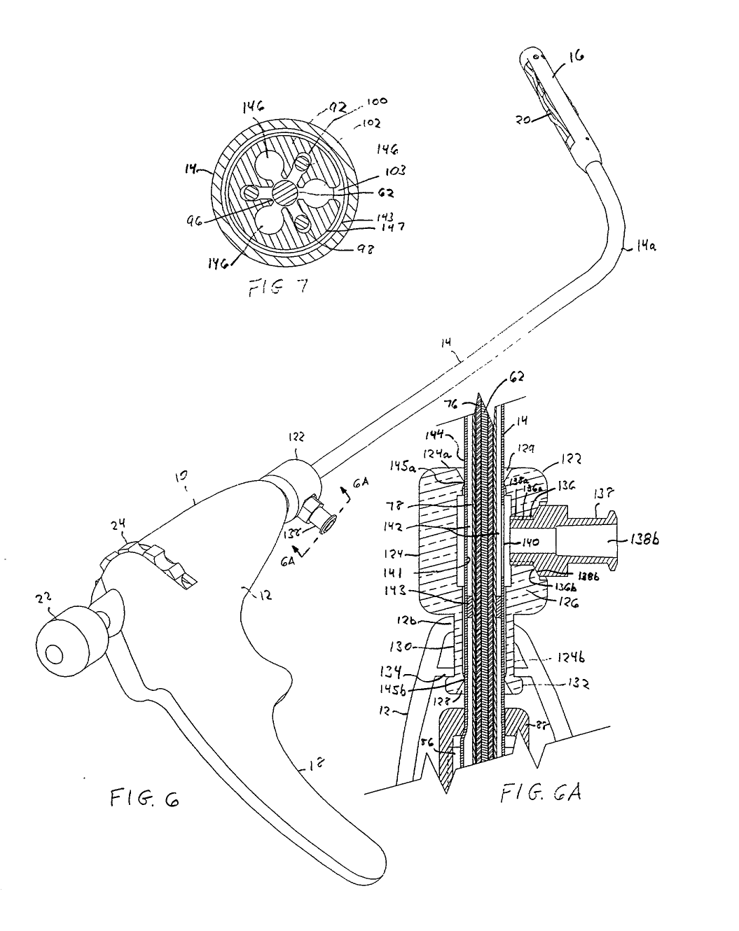

Referring to FIGS. 1A, 1B, 2, 2A-2C, and 3, the apparatus 10 of the present invention is shown having a housing 12, and a rigid, hollow shaft 14 extending from housing 12 to a distal end 16. The housing 12 has a body which may be shaped like a pistol having a handle portion 18. The shaft 14 is of a cylindrical cross-sectional shape with an outer diameter enabling the shaft to be inserted through the urethra of the penis of a male patient to locate the distal end 16 in the prostate gland of the patient (FIGS. 5A-5C). The shaft 14 is curved at the shaft's section 14a near the distal end 16 to allow that part of the shaft to pass by the curved bony diaphragm structure of the penis along which the urethra extends to the prostrate. The invention can also be used with other tubular tissue structures, e.g., uterus, esophagus, colon, or solid tissue structures, e.g., breast, muscle, neurologic, or orthopedic. The shaft may be otherwise curved, straight, or at other orientations depending on the particular path through the natural or surgical opening or channel through which the shaft and distal end must extend to access the particular tissue structure to be operated upon by apparatus 10. Although the apparatus 10 is described herein for use in human patients, it may also be used in animals with proper sizing of the shaft and distal end.

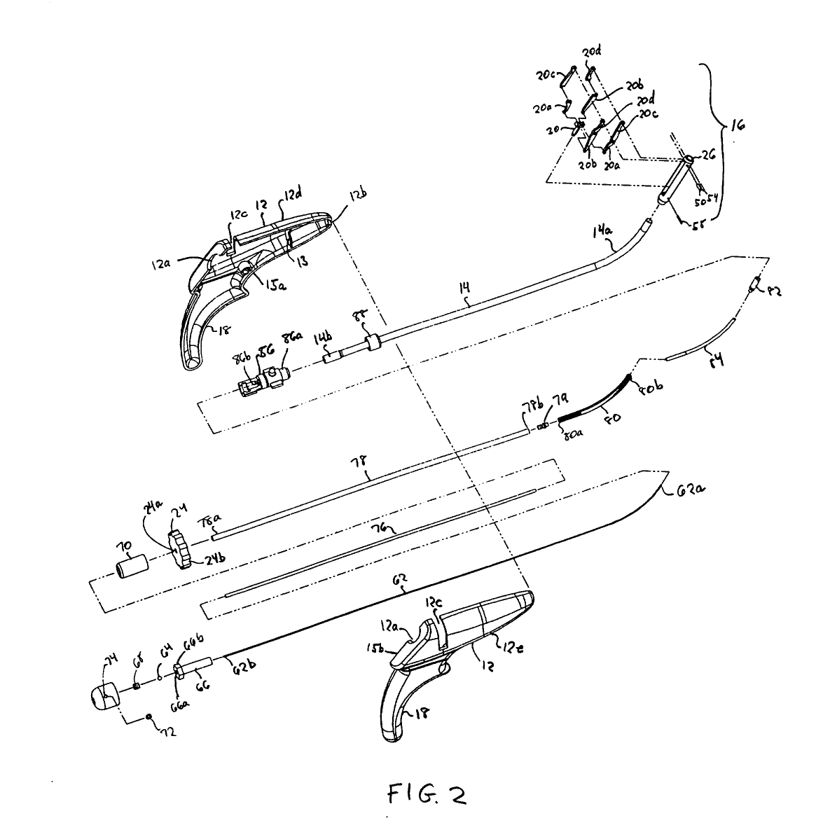

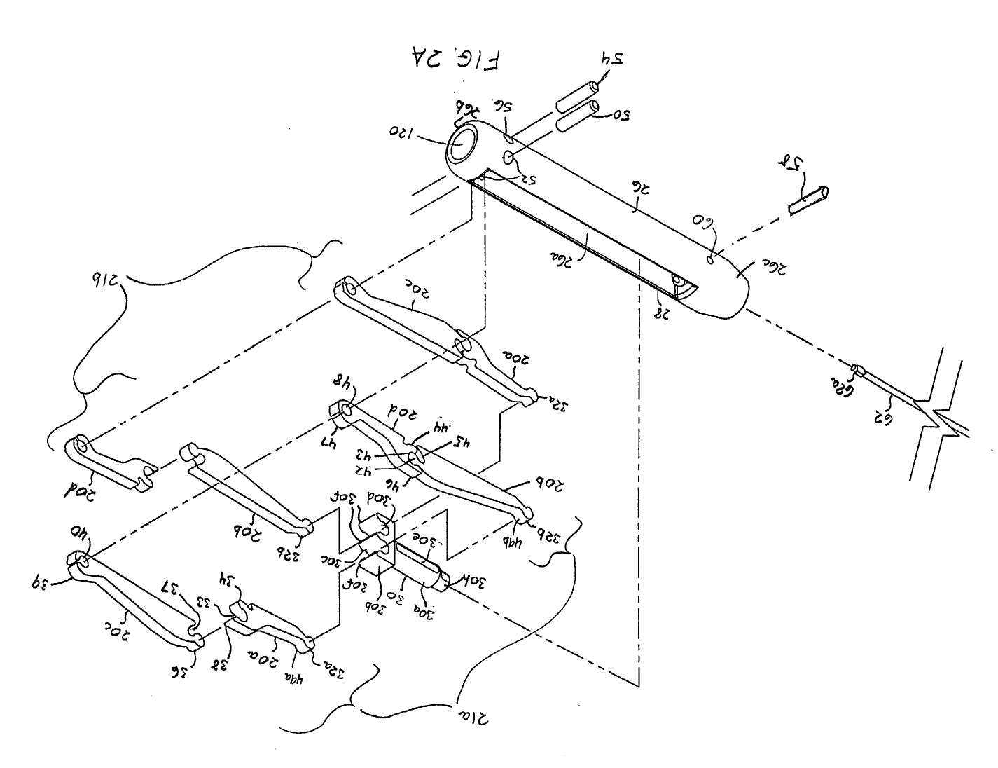

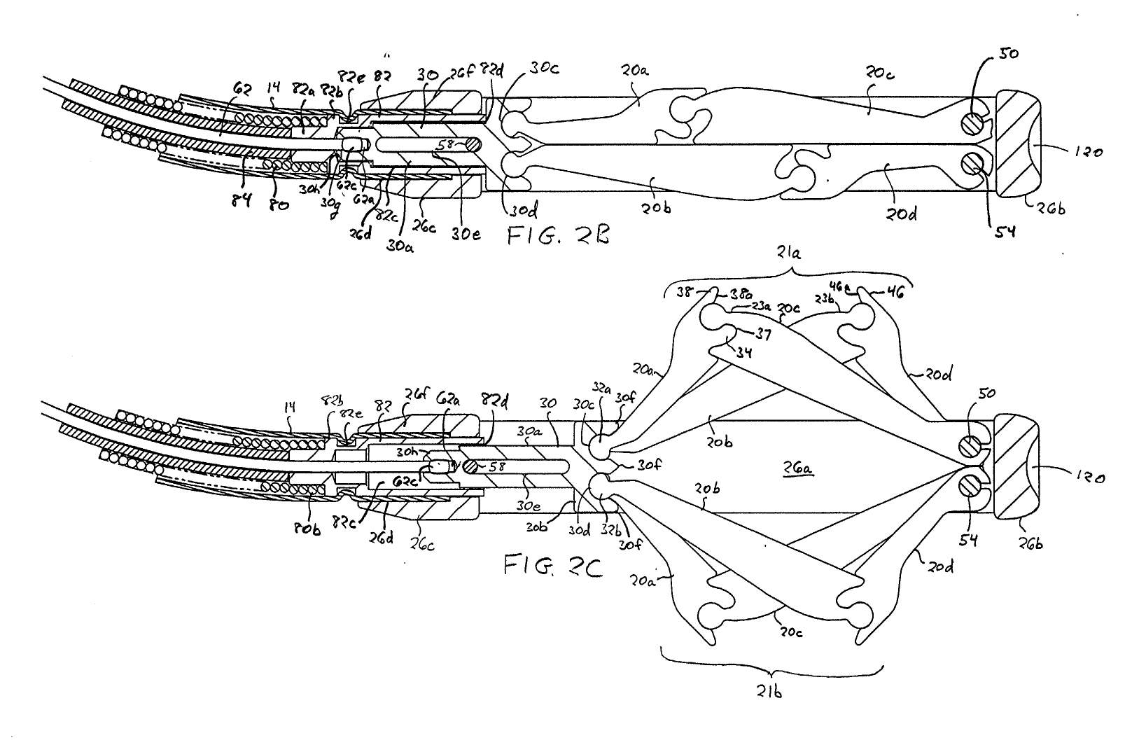

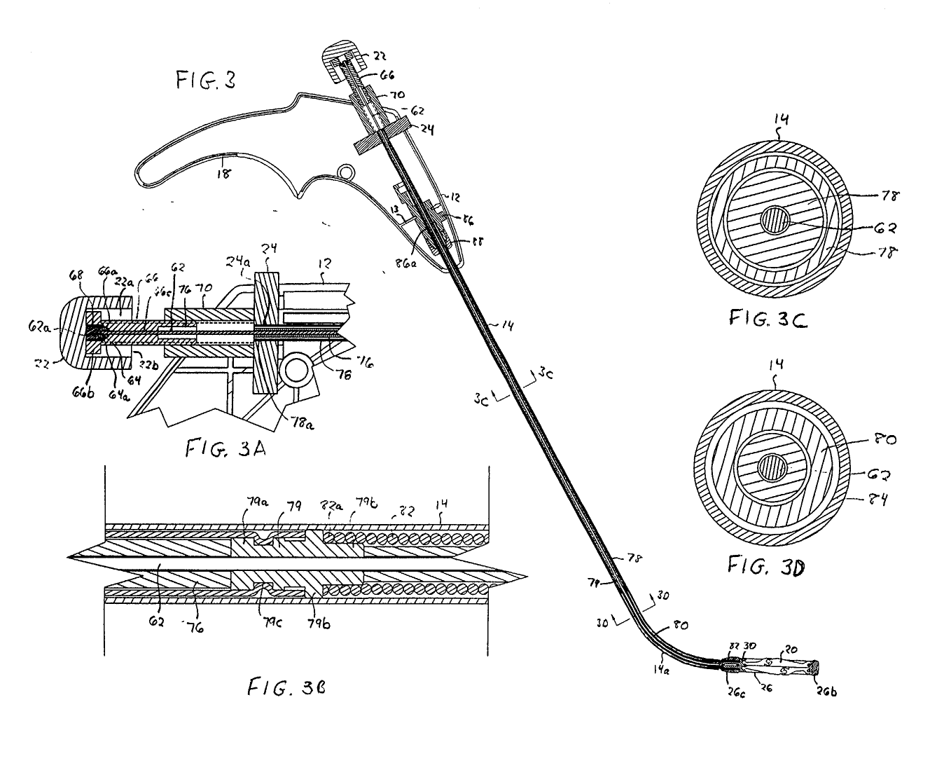

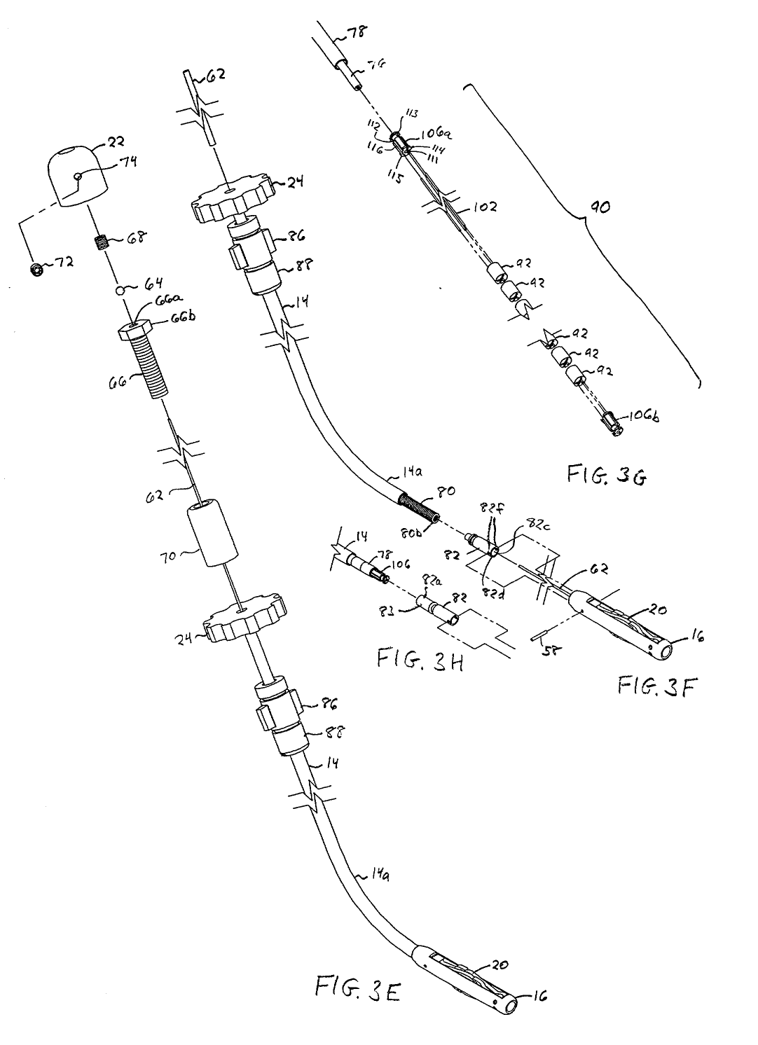

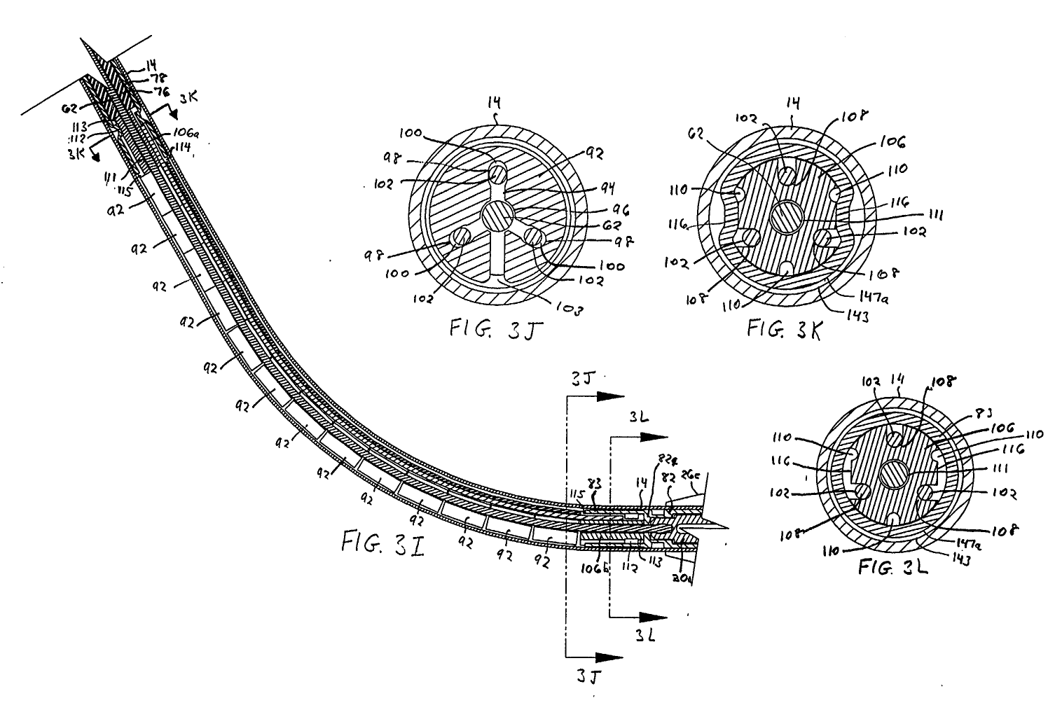

As best shown in FIGS. 2A-2C, distal end 16 has a distal cylindrical housing 26 providing a cavity 26a with two rectangular openings or windows 28 on opposite sides of the cavity, a top 26b, and an opening through its base 26c to the cavity. In the cavity 26, disposed for extension through each of the openings 28, is one of two sets 21a and 21b of two tissue engaging members 20. The tissue engaging members 20 are extendible from their retracted position, as shown in FIGS. 1A and 2B, to extend through the openings 28 opposite each other, as shown in FIGS. 1B and 2C. The tissue engaging members 20 are actuated responsive to a longitudinal drive mechanism, which extends from housing 12 through the shaft 14 to the distal end 16. The longitudinal drive mechanism is manually controlled at housing 12 by turning a cap 22 to rotate a turn-screw 66 threaded in a cylinder 70, in which one direction of rotation extends the tissue engaging members 20, and in the opposite direction retracts the tissue engaging members 20. A rotational drive mechanism is also provided through shaft 14 to rotate the distal end 16 upon the shaft. The rotational drive mechanism is manually controlled at housing 12 by a wheel 24 in which rotation of the wheel in either clockwise or counterclockwise direction is coupled through the shaft to rotate the distal end 16 in the same direction. The longitudinal and rotational drive mechanisms operate independent of each other and will be described later in more detail.

In each of sets 21a and 21b, the first of the two tissue engaging members 20 is formed by a proximal member 20a and a distal member 20c, and the second of the two tissue engaging members 20 is formed by a proximal member 20b and a distal member 20d. Each tissue engaging member 20 represents a hinged wing which is extendible radially through their respective opening 28 of the distal end. Proximal member 20a has a socket 33 which receives a curved member or shaft 36 extending from distal member 20c to form a hinge similar to a ball and socket joint. One side of socket 33 extends to form a finger 34 which may be received in an opening 37 of distal member 20c shaped to receive finger 34. Proximal member 20a has a barb 38 which extends from the other side of socket 33. Similarly, the proximal member 20b has a curved member or shaft 42 which is received in socket 43 of distal member 20d to form a hinge also similar to a ball and socket joint. One side of socket 43 extends to form a finger 44 which may be received in an opening 45 of proximal member 20b shaped to receive finger 44. Proximal member 20d has a barb 46 which extends from the other side of socket 43. Proximal member 20a and distal member 20d may be of the same first length, and proximal member 20b and distal member 20c may be of the same second length, where the first length is less than the second length. For example, the first length may be 0.498 inches, and the second length may be 0.849 inches from the center of each hinge.

In tissue engaging member set 21a, a hole 40 is provided at the end 39 of distal member 20c through which extends a pin 50 through two openings 52 in the sides of housing 26 near top 26b, and a hole 48 is also provided at end 47 of distal member 20d though which pin 50 also extends. Holes 40 and 48 are each optionally slotted along one side. In tissue engaging member set 21b, a pin 54 similarly extends through holes 40 and 48 through two openings 56 in the sides of housing 26 near top 26b. Each of pins 50 and 54 are adjacent the one of openings 28 through which their respective tissue engaging member sets 21a and 21b are extendible and retractable.

A plunger 30 is provided having a shaft section 30a which is linearly moveable in the base 26c of cylindrical housing 26 in a circular cross-section shaped cavity 82c of a coupler 82 which opens to cavity 26a and is shaped to receive shaft section 30a. The coupler 82 is received in circular cross-section shaped opening 26d of base 26c, as will be described later is more detail. The plunger 30 also has an upper section 30b having two parallel grooves which form sockets 30c and 30d aligned with pins 50 and 54, respectively. Shaft section 30a has a slot 30e extending in a parallel direction of such linear motion. In this slot 30e extends a guide pin 58 located through two openings 60 in base 26c of housing 26 to guide bi-directional movement of the plunger 30 in housing 26. At end 49a opposite socket 33 of proximal member 20a forms a curved member or shaft 32a, and at end 49b opposite pin 42 of proximal member 20b forms a curved member or shaft 32b. For tissue engaging member set 21a, curved members 32a and 32b of proximal members 20a and 20b, respectively, are received beside each other in plunger socket 30c and are rotatable therein. For tissue engaging member set 21b, curved members 32a and 32b of proximal members 20a and 20b, respectively, are received beside each other in plunger socket 30d and are rotatable therein. The walls 30f forming sockets 30c and 30d extend upwards to form fingers with tapered ends. This facilitates insertion of curved members 32a and 32b in one of sockets 30c and 30d for respective tissue engaging member sets 21a and 21b, such that the curved members 32a and 32b may inserted or removed from these sockets only at an angle not achievable when the distal end is fully assembled, thereby preventing the curved members 32a and 32b from falling out of their respective sockets during normal operation.

As the plunger 30 moves forward in housing 26 toward top 26b, curved members 32a and 32b rotate in socket 30c (for tissue engaging member set 21a) or 30d (for tissue engaging member set 21b), rotating curved members 36 and 42 of distal and proximal members 20c and 20b, respectively, in sockets 33 and 43 of proximal and distal members 20a and 20d, respectively, as distal members 20c and 20d rotate about pin 50 (for tissue engaging member set 21a) or 54 (for tissue engaging member set 21b), thereby extending outwards from the distal end 16 simultaneously both sets 21a and 21b of tissue engaging members 20. The degree of extension being controlled by the length of travel of the longitudinal drive mechanism and limited by fingers 34 and 44 of proximal and distal members 20a and 20d, respectively, being stopped by their full insertion into openings 37 and 45 of distal and proximal members 20c and 20b, respectively. As the plunger 30 moves backward in housing 26 toward base 26c, the above-described outward rotation of member 20a-d occur in the opposite direction, thereby retracting the tissue engaging members 20. The degree of retracting being controlled by the length of travel of the longitudinal drive mechanism and limited by the surface 38a of barb 3 8 of proximal member 20a abutting the surface 23b of distal member 20c, and the surface 46a of barb 46 of distal member 20d abutting the surface 25 of proximal member 20b. When fully retracted, the tissue engaging members 20 are substantially contained in housing 26, and may extend slightly beyond the outer diameter of cylindrical housing 26, as shown in FIG. 2B. The extension and retraction of the tissue engaging members is controlled by the longitudinal drive mechanism coupled to the plunger 30, which forms a part of this drive mechanism. The cylindrical housing 26, tissue engaging members 20, plunger 30, and pins 50, 54, and 58, may be of moldable plastic, stainless steel or other robust biocompatible material. The tissue engaging member 10 may be manufactured using electrical discharge machining (EDM) processes. Preferably, cylindrical housing 26 is a single component, but may be provided by several components bonded or welded together to form such housing. For example, the outer diameter of housing 26 maybe 0.315 inches.

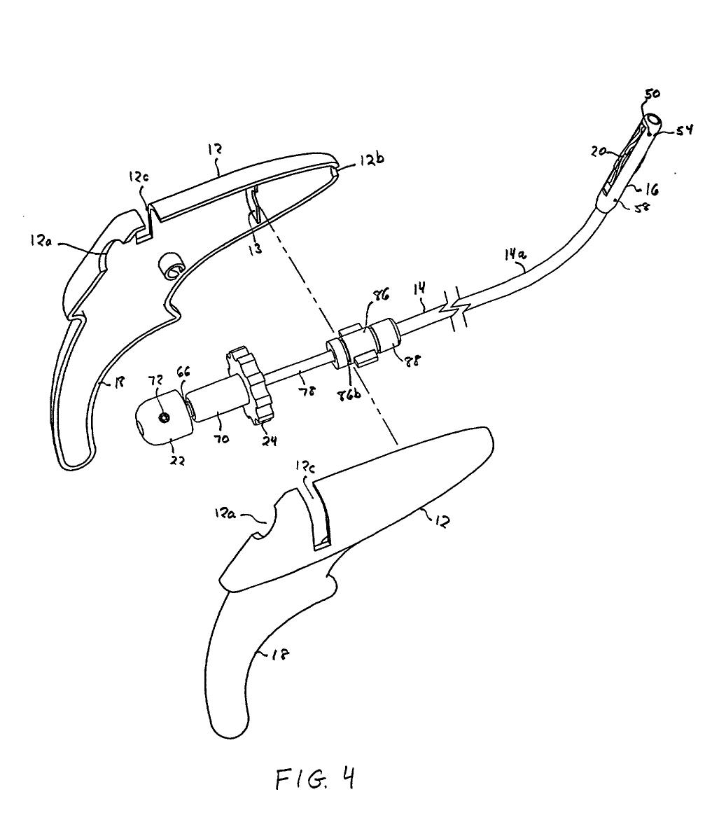

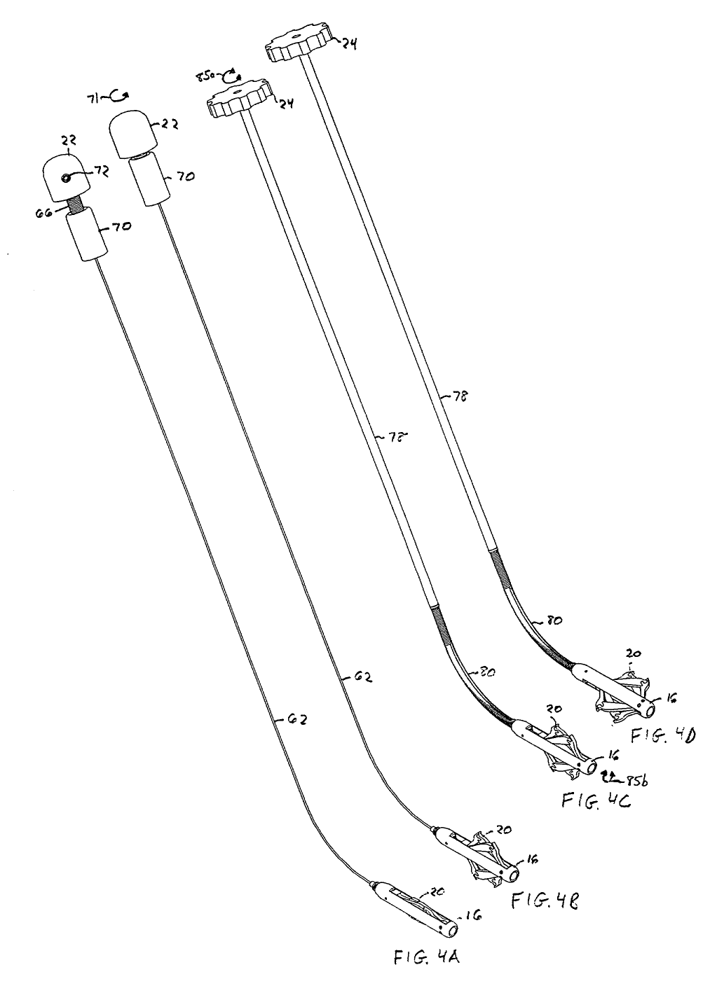

Referring to FIGS. 2B-C, and 3, the longitudinal drive mechanism includes a wire 62 which extends from housing 12 through shaft 14 to wire end 62a mounted into a hole 30g at end 30h of shaft section 30a of plunger 30, such that as distal end 16 rotates, the wire 62 although fixed allows the plunger 30 to rotate. The end 62a is deformed to form a swage 62c by flattening the sides of the wire by compression in a press. The swage 62c fits in hole 30g, and allows the wire 62 to be rotatable in hole 30f but is too large to pull out through hole 30f. The other end 62b of the wire 62 is attached to a ball 64, such as welded. A hole 64a may be provided in ball 64 into which the wire 62 is received prior to being attached to the ball. The ball 64 is then received in a recess 66a at the head 66b of a turn-screw 66, while the wire 62 extends from the ball 64 through a hole 66c extending through the turn-screw. A retainer member 68 is provided having an interior shaped to receive ball 64 and a threaded exterior enabling the retainer member to screw into recess 66a which in threaded to receive the retainer member. The interior of retainer member 68 and recess 66a define a socket in which the socket is rotatable about the ball 64. The turn-screw 66 is received in threaded cylinder 70 which is fixed in an opening 12a of housing 12. Cylinder 70 may be also molded as part of housing 12. Cap 22 has an interior cavity 22a and an opening 22b to the cavity shaped to receive the hexagonal shaped head 66b of turn-screw 66, such that a set screw 72 when received through threaded opening 74 of cap 22 locks the head 66b in cap 22. A dimple or recess may be provided on one or more of the hexagonal surfaces, such that when the hexagonal surface faces the screw 72 in cavity 22a the screw is received in the dimple. As shown in FIGS. 4A-4B, by turning cap 22, as indicated by arrow 71, the wire 62 is driven forward, pushing plunger 30 to extend the tissue engaging members 20 as described earlier, while turning the cap in the opposite direction retracts the wire 62 and the tissue engaging members 20. The wire may be a metal wire, and the other components of the longitudinal drive mechanism may be composed of metal, plastic, or other rigid materials. The wire 62 extends from turn-screw 66 to plunger 30 through multiple components which will now be described which principally provide for the rotational drive mechanism without applying rotation to the wire.

Referring to FIGS. 2, 2B-C, 3, and 3A-D and 3F, the rotational drive mechanism includes wheel 24 having a central opening 24a through which is attached one end 78a of a rigid inner tube 78, which is coupled at its other end 78b to one end 80a of a spring 80 through a first rotational coupler 79. Wheel 24 is located in an opening 12c of housing 12. FIG. 3C shows a cross-section of the shaft before reaching coupler 79, where the inner tube's outer diameter is slightly less than the inner diameter of shaft 14. A filler material or tube 76 surrounds wire 62 as it extend from wheel 24 to coupler 79 to avoid kinking, bending, or other undesirable flexing of the wire 62 due to driving or retracting wire 62 or rotation of the rotational drive mechanism. The filler material may be a lubricious, low friction material, such as a plastic tube, to fill the space between the wire and inner tube 78. As best shown in FIG. 3B, rotational coupler 79 has a first end 79a received into tube 78 until an annular lip 79b. The tube 78 is attached to coupler 79, such as staked, i.e., deformed, such as by a press, at one or more locations, into an annular groove 79c of coupler 79, so that rotation of tube 78 is coupled to coupler 79. End 80a of spring 80 is received onto other end 79d of coupler 79 until annular lip 79b and held thereupon by frictional engagement.

The spring 80 extends through the curved section 14a of the shaft 14 to its other end 80b into which is received one end 82a of a second rotational coupler 82 until an annular lip 82b, and held thereupon by frictional engagement. FIG. 3D shows a cross-section of the shaft 14 at curved section 14a, where the spring's outer diameter is less than the inner diameter of shaft 14. Another filler material or tube 84 of the same material as tube 76, but of a smaller outer diameter, fills the space between the spring 80 and the wire 62 between couplers 79 and 82.

Rotational coupler 82 couples the rotational drive mechanism to the distal end 16. As described earlier, the rotational coupler 82 has a cavity 82c and an opening 82d to the cavity with a circular cross-sectional shape which extends through opening 26d of base 26c of housing 26, and is shaped to receive the plunger shaft section 30a and end 30h of the shaft section, as shown in FIG. 2B. End 30h has a reduced diameter than the rest of the shaft section 30a, and the opening 82d is shaped accordingly to mate with the shaft section 30a when plunger 30 is retracted. The shaft 14 is staked at one or more locations, preferably three, at 120 degree intervals about the shaft 14 into an annular groove 82e to form a track along which the coupler 82 can be rotated while the shaft 14 remains fixed. The base 26c receives the end 14b of shaft 14 through its opening 26d along an annular ledge 26e locating the shaft 14 between the wall 26f of base 26c and the coupler 82. The outer surface of wall 26f may be tapered to facilitate removal of distal end from a patient's urethra or other channel or opening through with shaft 14 is extended, while the outer surface of top 26b is tapered to facilitate insertion of the distal end into the patient's urethra or other channel or opening through with shaft 14 is extended. The base 26c of the distal end can rotate while the shaft 14 remains fixed upon ledge 26e. Guide pin 58 when located through slot 30e also extends through two openings 82f (FIGS. 3 and 3F) in the sides of coupler 82, so that as coupler 82 rotates such rotation is translated by guide pin 58 to the distal end 16. Thus, the rotational drive mechanism provides for coupling the rotation from wheel 24 through tube 78, coupler 79, spring 80, coupler 82, and pin 58 to distal end 16. The wheel 24 may be of plastic or metal, while coupler 79, spring 80, coupler 82, and pin may be composed of metal or other biocompatible material. The wire 62 of the longitudinal drive mechanism passing through the parts of the rotational drive mechanism, including tube 78, coupler 79, spring 80, and coupler 82, into hole 30g of plunger 30, but without coupling rotation of the drive mechanism to wire 62. The entire distal end 16 can be rotated irrespective of the extension position (retracted, intermediate, or full extension) of the tissue engaging members 20. FIGS. 4C-D show an example of the distal end 16 rotates as indicated along one direction of bi-directional arrows 85a and 85b, such as 90 degrees.

The shaft 14 extends in housing 12 through an opening 12b of the housing 12, and has a D-shaped end 14c, which is received into a D-shaped opening of an adaptor 86. A nut 88 having an interior threaded surface retains the shaft 14 onto the threaded end 86a of the adapter. An opening 86b extends through the adapter 86, and rigid tube 78, filler 76, and wire 62 passes through this opening 86b, as shown in FIG. 3. Tube 78 is attached to wheel 24, and wire 62 coupled to cap 22, as described earlier, to provide controllers of the rotational and longitudinal drive mechanism, respectively. The housing may be made of molded plastic of two halves 12d and 12e which mate along edges 15a and 15b, respectively, to form housing 12, as shown in FIG. 4. An internal flange or rib 13 in each half 12d and 12e supports adapter 86 along an annular groove 86b of adapter 86. The two halves 12d and 12e are attached to each other along their edges 15a and 15b with the assembled distal end 14, shaft 14, and the longitudinal and rotational drive mechanisms. The shaft may be a cylindrical stainless steel metal tube curved at an angle near the distal end 16. For example, the shaft may have an outer and inner diameter of 0.203 and 0.199 inches, respectively. The curvature of this angle may be, for example a radius of 120 degrees off the occluded angle. The adapter 86 and nut 88 may be made of metal or molded plastic.

Spring 80 represent a flexible member for coupling rotation through the curved section 14a, but other flexible members may also be used to couple rotation, as may be provided by the assembly 90 shown in FIGS. 3G-3L. Assembly 90 has multiple rigid sections 92 each with a spoked interior cavity 94 with three spokes 98 and a central channel 96, where each of the spokes ends at a channel 100. The rigid sections are stacked through shaft section 14a, but are moveable therein along three guide wires 102, one in each channel 100 of the three spokes 98. For purposes of illustration thirteen rigid sections 92 are shown in FIG. 3I, however other number of sections 92 may be used, such as sixteen. End sections 106a and 106b are provided before the first and after the last rigid section 92, respectively. Each of end sections 106a and 106b have three outer grooves 108 providing channels, one for each of wires 102, three outer alignment grooves 110, a central channel 111 for wire 62, an annular groove 112 at one end 113, and an edge 114 at the other end 115 which faces its respective adjacent rigid section 92. Along two sides of each of the end sections 106a and 106b, recesses 116 are provided. Sections 92, 106a, and 106b may be made of stainless steel, and may be manufactured on EDM machinery. The outer diameter of the rigid sections 92 are slightly less than the inner diameter of shaft 14, such that the sections 92 can rotate in the shaft. For example, the length of each of sections 92 may be 0.200 inches with an outer diameter of 0.156 inches, and the length of end sections 106a and 106b may be 0.285 inches with an outer diameter along edge 114 approximately the same as the outer diameter of sections 92.

In the embodiment of apparatus 10 using assembly 90, the rotational coupler 79 and spring 80 are not used, and rotational coupler 82 has a cylindrical wall 83 extending from a wall 82g at lip 82b providing an open end 82a. End section 106a is received into the end 78b of rigid tube 78, and attached to the tube, such as by staking. End section 106b is received into the cylindrical wall 83 at end 82a of coupler 82, and attached thereto, such as by welding. Wires 102 are attached only at end section 106a, such as by welding, and are slidable in their respective channels 100 of rigid sections 92, and slidable also at opposite end section 106b where they are not attached. By attaching wires 102 only at the proximal end in section 106a, but not in rigid sections 92 or end section 106b, rotation is coupled by the rotational drive mechanism from wheel 24 to turn distal end 16. However, turning distal end 16 will not couple back rotation through the rotational drive mechanism, but will bind one or more of the linkages provided by sections 92 and thus act as a anti-rotation lock. In this embodiment of the rotational drive mechanism of apparatus 10, rotation is coupled from wheel 24 to tube 78 through end section 106a, along successive rigid sections 92 to end section 106a, and then to coupler 82 and pin 58 to rotate distal end 16. Assembly 90 may be preferred over use of spring 80 to couple rotation through shaft section 14a, since the spring may require additional rotation at its end 80a to translate rotation to end 80b due to compression of the spring, thereby reducing responsiveness of the rotational drive mechanism, and could store energy in the spring when rotated causing undesirable counter rotational force. Assembly 90 further makes the rotation of the distal end more responsive to rotation of wheel 24. Other mean, such as a flexible tube may also be used to translate rotation through curved shaft section 14a.

Referring to FIGS. 5A-5C, a schematic of the male pelvic anatomy is shown for illustrating the method for using apparatus 10 in a prostatectomy procedure. FIG. 5A shows the male pelvis anatomy prior to insertion of apparatus 10. Apparatus 10 is inserted transurethral by an operator, e.g., surgeon, into the penis 112 in which the curved section 14a of the shaft 14 allows the shaft to travel along the urethra 114 passed the bony diaphragm structure 115 of the pelvis until the distal end 16 is located in the prostate 116. Once in the prostate 116, the cap 22 is rotated by the surgeon to drive the tissue engaging members 20 to extend into the prostate, as shown in FIG. 5B. Barbs 38 and 46 on the tissue engaging members facilitate gripping of the prostate 116 onto the distal end 16. Although the tissue engaging members 20 are shown fully extended, they may be extended to any desired degree by the surgeon until full extension. The prostate's position can then be manipulated as needed to facilitate prostatectomy. The positioning of the prostate is provided under control of the surgeon, such as, raised or lowered by adjusting the tilt angle of the shaft 14 with respect to the patient's body, pulled or pushed by changing the extent of the shaft 14 passing through the urethra 114 (i.e., slightly pushing or pulling housing 12), and bi-directionally rotated using wheel 24. The outer edge 24b of wheel 24 may be grooved to facilitate the surgeon in gripping the wheel. In this manner, the surgeon can position the prostate 116 to expose and apply tension to the tissue at the anterior side of the prostate, and thereby locate the area or zone of dissection and proceed to mobilize (or cut the surrounding tissue of) the prostate at its anterior side. The prostate's position may then be further manipulated with apparatus 10 to facilitate exposing and placing under tension the area or zone of dissection and proceed to mobilize the tissue (or cut the surrounding tissue) along the posterior side and both lateral sides of the prostate 116. Once the prostate has been dissected, turning cap 22, as shown in FIG. 5C, retracts the tissue engaging members 20. The prostate can then be removed from the patient and the urethra sutured to the bladder 118. A recessed circular dimple 120 or opening extending to cavity 26a (FIGS. 2A-C) may be provided on top 26b of the distal end 16 to aid in centering another instrument with respect to apparatus 10. The apparatus 10 thus provides a surgical instrument, which is useful in either open surgery, perineally, or laproscopic prostatectomy, but may also be used in other surgical procedures to manipulate tissue structures other than the prostate via a natural or surgical opening or channel in the body of a patient. The shape of housing 12 facilitates ease of single hand use by the surgeon, whereby the thumb can move cap 22, the wheel 24 can be moved by the index finger, while other fingers grip the handle portion 18. Such control of the prostate's position enables precise dissection thereby minimizing the risk of damage to the neurovascular bundles and other tissue about the prostate.

Referring to FIGS. 6 and 6A, another embodiment of the invention is shown for enabling passage of gasses or fluids between distal end 16 of apparatus 10 and a port 122 coupled to the shaft 14. The port 122 has a cylinder 124 having a chamber 126, and two openings 128 and 129 to the chamber 126. Each of the openings 128 and 129 extends through one of the ends 124a and 124b, respectively, of the cylinder 124. At end 124b, opening 128 extends through a smaller diameter extension 130 of the cylinder 124 to an annular lip 132. Near housing 12, the shaft 14 extends through opening 129, chamber 126, opening 129, and extension 130 of the cylinder 124, such that extension 130 is received through opening 12b of housing 12, and an internal flange or rib 134 in each housing half 12d and 12e supports port 122 about lip 132, as shown in FIG. 6A. The opening 12b is of a diameter for receiving extension 130 of the cylinder 124. A bore or opening 136 is provided along the side of the cylinder to chamber 126. This opening 136 is threaded along surfaces 136a to enable a threaded fitting member 138 to be screwing along surfaces 136a into opening 136. Surface 136b along the opening 136 may mate with surface 138a of the fitting member 138. Fitting member 138 has an opening or passageway 138b extending therethrough to the chamber 126 to enable passage of fluids or gasses to or from chamber 126.

Facing opening 138b is an opening 140 through the shaft 14 to a channel (or region) 142 formed between the outer surface of rigid tube 78 and the inner surface 141 of shaft 14. In this channel 142 is a sealing ring 143 defining a closed end of the channel. At each end 124a and 124b of the cylinder 124 is a seal formed by the cylinder against the outer surface 144 of shaft 14. One seal is provided by an annular flap or ridge 145a extending inward in opening 129, and another seal is provided by an annular flap of ridge 145b extending inward in opening 128. The cylinder 124 may be of a material, such as polyethylene, polypropylene, Teflon, or other material sufficiently compliant to form these seals at 145a and 145b. The fitting member 138 may be a luer type fitting, and may be made of material, such as plastic or metal.

Inside shaft 14, channel 142 forms multiple channels or passageways in this embodiment, since fluids and gasses may pass through spaces about tubes, couplers, or wire of the components within the shaft 14 to the distal end 16. Preferably to provide additional capacity for fluids and gasses, the exterior surfaces of rigid tube 78, coupler 79, and spring 80 may each be of a even smaller outer diameter than the inner diameter of shaft 14 earlier described. When assembly 90 is used instead of spring 80 in apparatus 10 to translate rotation through shaft section 14a, each of the rigid sections 92 may have channels 146 from additional spokes 98 in interior cavity 94, as shown in FIG. 7. Grooves 110 (FIG. 3K) along end sections 106a and 106b may be used to provide channels for fluids or gasses. Fluids or gases may also pass through other available space about wires 62 and 102 in cavity 94, opening 103 to cavity 94, and between the exterior surface 147 of rigid sections 92 and interior surface 143 of shaft 14, and the exterior surface 147a of end sections 106a and 106b (FIGS. 3L and 3K) and interior surface 143 of shaft 14.

To facilitate passage of fluids or gasses to cavity 26a of the distal cylindrical housing 26, and thus to the area or region of the patient where the distal end 16 of the apparatus 10 is located, additional holes may be provides through components of the distal end 16. For example, when the apparatus 10 uses spring 80, this may be achieved by providing holes through annular lip 82b of coupler 82 to its cavity 82c. In the case where the apparatus 10 uses assembly 90, one or more holes may be provided through wall 82g or lip 82b of the coupler 82 to cavity 82c. Further, the interior diameter of cavity 82c may be sized smaller than the outer diameter of plunger shaft 30a and end 30h to provide additional space between the plunger 30 and the interior of cavity 82c for passage of fluids or gases. Also, the plunger's upper section 30b and/or shaft 30a may have holes extending therethrough, such that the fluid or gas may pass between cavity 82c and cavity 26a.

In operation, fluid or gasses may be inserted or removed from the area of a patient's body where the distal end is located, such as the site of tissue engagement, through cavity 26a, shaft 14, and port 122. This may be useful to remove bodily fluids or gasses from area of tissue dissection, such as by suction applied through port 122, from, for example, a vacuum pump via connecting tubing (not shown) to fitting member 138. Similarly, a fluid or gas may be inserted via appropriate connector to a source of such fluid of gas.

From the foregoing description, it will be apparent that an improved apparatus and method for manipulating tissue has been provided. Variations and modifications in the herein described an apparatus and method in accordance with the invention will undoubtedly suggest themselves to those skilled in the art. Accordingly, the foregoing description should be taken as illustrative and not in a limiting sense.

1. An apparatus for manipulating tissue in the body of a patient comprising:
a housing, a shaft extending from the housing, and a distal end coupled to said shaft;
said distal end having tissue engaging members extendible into said tissue from a retracted position at said distal end;
means for driving said tissue engaging members to extend into said tissue and to return to said retracted position; and
means for rotating said distal end, wherein when said tissue engaging member engage said tissue, said tissue is positionable by movement of said housing coupled to said distal end by said shaft, and positionable by movement of said rotating means.

2. The apparatus according to claim 1 wherein said tissue represent the prostate gland of a male patient, and said shaft is positionable through the urethra of a male patient to locate said distal end in the prostate of the patient.

3. The apparatus according to claim 2 wherein said shaft has a curved section to facilitate insertion through the urethra to the prostate.

4. The apparatus according to claim 1 wherein said shaft is insertable through a natural or surgical opening or channel in the body of the patient to said tissue.

5. The apparatus according to claim 4 wherein said shaft is of a shape enabling passage of said shaft to said tissue through said opening or channel.

6. The apparatus according to claim 1 wherein said driving means and rotating means operate independent of each other.

7. The apparatus according to claim 1 further comprising means for controlling said driving means and rotating means from said housing.

8. The apparatus according to claim 1 wherein said tissue engaging members each represent by two members, and a hinge coupling said members for rotation about said hinge when said tissue engaging member extends and retracts, in which said members are rotationally mounted in said distal end for rotation along said hinge in response to said driving means.

9. The apparatus according to claim 8 wherein said driving means comprises:
a plunger and means for mounting to said plunger a first of said members of said tissue engaging members for rotational movement,said plunger being linearly moveable in said distal end;
a wire extending through said shaft and coupled to said plunger; and
means for controlling the extension or retraction of said wire to move said plunger and rotate said first member and members about said hinge of each of said tissue engaging members, to extend or retract said tissue engaging members.

10. The apparatus according to claim 9 wherein said wire has first and second ends and said first end is coupled to said plunger, and said controlling means comprises:
a ball attached to said second end of said wire;
a turn-screw having a socket coupled to said ball and said wire extends from said socket to said plunger, in which said rotation of said socket is decoupled from said ball;
a cylinder through which said turn-screw is mounted for rotation; and
mean for rotating said turn-screw which drives said wire to extend and retract said wire.

11. The apparatus according to claim 1 wherein said shaft has a curved section near said distal end, and said rotating means comprises a rigid member coupled for rotation to a flexible member, in which rigid member extends from said housing through said shaft, said flexible member extends through said curved section of said shaft, and said flexible member is mounted for rotating said distal end upon said shaft.

12. The apparatus according to claim 11 wherein said rigid member is attached to a wheel in said housing which is rotatable to translate rotation to said rigid member and said flexible member to rotate said distal end.

13. The apparatus according to claim 11 wherein said flexible member represents a spring.

14. The apparatus according to claim 11 wherein said flexible member comprises:
a plurality of successive rigid sections having channels; and
a plurality of wires which extend through said channels to translate rotation through said rigid sections.

15. The apparatus according to claim 1 further comprising a port and means for passing one of a liquid, gas, or both liquid and gas, along said shaft between said port and said distal end.

16. The apparatus according to claim 1 wherein said rotating means comprises means for coupling rotation through said shaft to rotate said distal end.

17. The apparatus according to claim 1 wherein said rotating means comprises means for coupling rotation through said shaft to rotate said distal end, and means for preventing said distal end from coupling rotation back to said coupling means.

18. A method for manipulating the prostate using an instrument having a shaft extending to a distal end having members capable of engaging the prostate, said method comprising the steps of:
inserting the shaft and distal end through the urethra of a patient to locate the distal end in the prostate;
extending the members to engage and hold the prostate onto the distal end; and
positioning the prostate to expose and tension tissue to aid in the dissection of tissue which enables removal of the prostate from the patient, while minimizing damage to surrounding tissue.

19. The method according to claim 18 wherein said positioning step further comprising the step of positioning the prostate by adjusting the tilt angle of the shaft.

20. The method according to claim 18 wherein said positioning step farther comprising the step of positioning the prostate by changing the extent of the shaft passing through the urethra.

21. The method according to claim 18 wherein said positioning step further comprising the step of bi-directional rotating the distal end.

22. The method according to claim 18 further comprising the steps of:
retracting the members at least substantially into said distal end; and
removing said shaft and distal end from the urethra of the patient.

23. The method according to claim 18 wherein said shaft has a port and said method further comprises the step of passing one of a liquid, gas, or both liquid and gas, along said shaft between said port and said distal end.
